(19) United States
(12) Patent Application Publication
(10) Pub. No.: US 2023/0012268 A1
(43) Pub. Date: Jan. 12, 2023

(54) COMPOSITIONS AND METHODS FOR TREATMENT OF INFLAMMATION WITH STEROIDS AND A MODULATOR

(71) Applicant: BUZZELET DEVELOPMENT AND TECHNOLOGIES LTD., OR-AKIVA (IL)

(72) Inventors: AHARON EYAL, JERUSALEM (IL); NOA RAZ, GIZO (IL)

(21) Appl. No.: 17/784,682

(22) PCT Filed: Dec. 13, 2020
(86) PCT No.: PCT/IB2020/061866

§ 371 Date: Jun. 13, 2022

Related U.S. Application Data
(60) Provisional application No. 62/947,552, filed on Dec. 13, 2019.
(60) Provisional application No. 62/947,550, filed on Dec. 13, 2019.

(51) Int. Cl. A61K 31/05; A61P 37/00

(52) U.S. Cl. CPC A61K 31/05; A61P 37/00

(57) ABSTRACT

Provided are pharmaceutical compositions comprising a pharmaceutically effective amount of a steroid and a pharmaceutically effective amount of at least one modulator selected from the group consisting of a transient receptor potential (TRP) channel modulator and a proliferatior-activated receptor (PRP) modulator, wherein cannabidiol is not present as the sole modulator, and uses thereof for treating a condition or a symptom thereof, wherein said condition involves inflammation, an immune disease, an autoimmune disease or a combination thereof.

### CROSS-REFERENCE TO RELATED APPLICATIONS

The present application gains priority from U.S. Provisional Patent Application Ser. No. 62/947,550 filed 13 Dec. 2019, and U.S. Provisional Patent Application Ser. No. 62/947,552 filed 13 Dec. 2019, both of which are incorporated by reference as if fully set-forth herein.

### FIELD OF THE INVENTION

The field of art to which this invention generally pertains is pharmaceutical compositions, and more specifically to pharmaceutical compositions and uses thereof for treating a condition involving inflammation, an immune disease, an autoimmune disease or a combination thereof comprising a steroid and a modulator selected from the group consisting of a PPAR modulator and a TRP channel modulator.

### BACKGROUND OF THE INVENTION

Steroids are administered in treating multiple indications. Steroid administration leads in many cases to undesired side effects. There is a strong need for pharmaceutical compositions and treatment methods that reduce the amount of steroids used and the related side effects.

### SUMMARY OF THE INVENTION

According to an aspect of some embodiments of the present invention, there is provided a pharmaceutical composition comprising a carrier and a pharmaceutically effective amount of a steroid and a pharmaceutically effective amount of at least one modulator selected from the group consisting of a transient receptor potential (TRP) channel modulator and a peroxisome proliferator-activated receptor (PPAR) modulator, wherein cannabidiol is not present as the sole modulator.

According to an aspect of some embodiments of the present invention, there is provided a pharmaceutical preparation comprising the pharmaceutical composition as disclosed herein.

According to an embodiment, the pharmaceutical composition or the pharmaceutical preparation as disclosed herein is for use in treating a condition or a symptom thereof wherein said condition involves inflammation, an immune disease, an autoimmune disease or a combination thereof.

According to an aspect of some embodiments of the present invention, there is provided a method for treating in a subject in need a condition or a symptom thereof wherein said condition involves inflammation, an immune disease, an autoimmune disease or a combination thereof, comprising administering to said subject a pharmaceutically effective amount of the composition or the pharmaceutical preparation as disclosed herein.

### DETAILED DESCRIPTION OF THE INVENTION

The present invention, in at least some embodiments thereof, relates to pharmaceutical compositions comprising a pharmaceutically effective amount of at least one steroid and a pharmaceutically effective amount of at least one modulator selected from the group consisting of a transient receptor potential (TRP) channel modulator and a proliferatior-activated receptor (PRP) modulator, wherein cannabidiol is not present as the sole modulator, and uses thereof.

Known side-effects caused by administration of a steroid to a subject, as compared to those present in the same subject in the absence of administration of a steroid, include, but are not limited to reduced immune function/increased susceptibility to infection; increased blood pressure; weight gain (particularly around the area of the abdomen) and/or increased appetite; increased blood sugar levels; changes in appearance of the skin and/or the hair; osteoporosis; stomach aches; muscle pains; increased intraocular pressure, such as leading to glaucoma; sleep disturbances; nervousness; hyperactivity; inhibition of growth in children. The present inventors have found that administration of a steroid together with at least one TRP channel modulator and/or PRP modulator, reduces the amount of steroid necessary to provide the same effect in the absence of the modulator, thereby reducing the side-effects associated with administration of the steroid.

As used herein, the term “modulator” refers to a substance that binds to and alters the activity of a receptor. A modulator may include, for example, a ligand, an activator, a full agonist, a partial agonist, an antagonist, an inverse agonist, a positive allosteric modulator and a negative alloseric modulator.

As used herein the term “treating” includes preventing, curing, ameliorating, mitigating, and reducing the instances or severity of a condition or a symptom thereof.

As used herein, the term “chronically administering” means administering daily for a continuous time period, such as at least a week, at least 10 days, at least two weeks, at least three weeks, at least one month, at least two months, at least three months, at least four months, at least five months or at least six months.

As used herein, the term “cannabidiol” (or “CBD”) is intended to encompass both cannabidiolic acid (CBDa) and cannabidiol (or CBD) unless indicated otherwise.

Unless indicated otherwise, the term “cannabinoid” as used herein refers to a compound that affects the endocannabinoid system and includes the acid form and the decarboxylated form of the compound, or combinations thereof. Cannabinoids are agonists or antagonists to receptors in the endocannabinoid system.

As used herein, the term “THC’ refers to THCa (tetrahydrocannabiniolic acid) and/or to THC (tetrahydrocannabiniol) unless indicated otherwise. As used herein, the term “CBG” refers to CBGa (cannabigerolic acid) and/or to CBG (cannabigerol) unless indicated otherwise. As used herein, the term “CBN” refers to CBNa (Cannabinolic acid) and/or to CBN (cannabinol) unless indicated otherwise. As used herein, the term “CBC” refers to CBCa (cannabichromenic acid) and/or to CBC (Cannabichromene) unless indicated otherwise. As used herein, the term “CBL” refers to CBLa (Cannabicycol acid) and/or to CBL (Cannabicyclol) unless indicated otherwise. As used herein, the term “THCV” refers to THCVa (tetrahydrocannabivarin acid) and/or to THCV (tetrahydrocannabivarin) unless indicated otherwise. As used herein, the term “CBDV” refers to CBDVa (cannabigerovarin acid) and/or to CBDV (cannabidivarin) unless indicated otherwise.

As used herein, the term “terpene” refers to both terpenes and terpenoids.

Unless indicated otherwise, percent is weight percent and ratio is weight/weight ratio. Unless indicated otherwise, weight ratio means the ratio between weight content.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

As used herein, when a numerical value is preceded by the term “about”, the term “about” is intended to indicate +/−10% of that value.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

According to an aspect of some embodiments of the present invention, there is provided a pharmaceutical composition comprising a carrier and a pharmaceutically effective amount of a steroid and a pharmaceutically effective amount of at least one modulator selected from the group consisting of a TRP channel modulator and a PPAR modulator, wherein cannabidiol is not present as the sole modulator.

According to an embodiment, cannabidiol is not present in the composition.

According to an embodiment, cannabidiol is present in the composition together with at least one additional modulator. According to an embodiment, the at least one additional modulator is selected from the group consisting of a TRP channel modulator and a PPAR modulator or a combination thereof.

According to an embodiment, said at least one modulator comprises at least one TRP channel modulator.

According to an embodiment, said at least one modulator comprises at least two TRP channel modulators.

According to an embodiment, said said at least one modulator comprises modulators of at least two different TRP channel modulators, such as modulators of at least two different TRP channels or at least two different modulators of a same TRP channel.

According to an embodiment, said at least one TRP channel modulator comprises a TRPV channel modulator, such as a TRPV1 channel modulator.

According to an embodiment, said at least one TRP channel modulator is selected from the group consisting of a TRPA1 modulator and a TRPM8 modulator, or combinations thereof.

According to an embodiment, said at least one modulator comprises a PPAR modulator.

According to an embodiment, said at least one PPAR modulator comprises a PPAR-γ modulator.

According to an embodiment, said at least one PPAR modulator comprises at least two PPAR modulators.

According to an embodiment, said at least one PPAR modulator comprises modulators of at least two different PPARs.

According to an embodiment, said at least one PPAR modulator comprises two different modulators of a same PPAR.

According to an embodiment, said at least one modulator comprises a TRP channel modulator and further comprises at least one peroxisome proliferator-activated receptor (PPAR) modulator.

According to an embodiment, said at least one modulator comprises a TRP channel modulatoar and further comprises at least one PPAR-γ modulator.

According to an embodiment, said at least one modulator comprises a TRP channel modulator and further comprises at least two PPAR modulators.

According to an embodiment, said at least one modulator comprises a TRP channel modulator and further comprises modulators of at least two different PPARs.

According to an embodiment, said at least one modulator comprises a TRP channel modulator and further comprises at least two modulators of a same PPAR.

According to an embodiment, said pharmaceutical composition comprises at least two TRP channel modulators and at least two PPAR modulators.

According to an embodiment, said pharmaceutical composition further comprises cannabidiol.

According to an embodiment, said pharmaceutical composition further comprises tetrahydrocannabinol.

According to an embodiment, said pharmaceutical composition further comprises cannabidiol and tetrahydrocannabinol, wherein a cannabidiol to tetrahydrocannabinol weight per weight ratio is greater than 3:1, such as at least 3:1, at least 4:1, at least 5:1, at least 6:1, at least 7:1, at least 8:1, at least 9:1 or at least 10:1.

According to an embodiment, said at least one modulator is a cannabinoid.

According to an embodiment, said at least one modulator is a terpene.

According to an embodiment, said at least one PPAR modulator is selected from the group consisting of Beta Caryophyllene, Pinene, Carvacrol, Geraniol, Farnesol, geranylgeraniol, Phytol, Phytanic acid, Abietic acid, Auraptene, bixin, norbixin, Amorfrutins, Formononetin, (−)-Catechin, THC, (9S,13R)-12-Oxo-phytodienoic acid, Hydroxy unsaturated fatty acids, Commipheric acid, Kaempferol-3-O-β-glucopyranoside, Citral, Alkamides, Tocotrienols, Deoxyelephantopin, Acylated flavonol glycosides, Kaempferol, quercetin, Genistein, 5′-Formylglabridin, (2R,3R)-3,4′,7-trihydroxy-3′-prenylflavane, echinatin, (3R)-2′,3′,7-trihydroxy-4′-methoxyisoflavan, kanzonol X, kanzonol W shinpterocarpin, licoflavanone A, glabrol, shinflavanone, gancaonin L, glabrone, Licochalcone E, Flavonoids and 3-arylcoumarins, Meranzin, Magnolol, honokiol, Lunularin, Cucurbitane-type triterpene glycosides, Polyacetylenes, Biochanin A, Ginsenoside 20(S)-protopanaxatriol, ginsenoside Rb₁, Oleanonic acid, Pseudolaric acid B, Daidzein, Amorphastilbol, Carnosic acid, carnosol, 12-O-methyl carnosic acid, α-linolenic acid, α-Linolenic acid, linoleic acid, naringenin, Saurufuran A, Isosilybin A, Gallotannins, Isoflavones, Ellagic acid, epicatechin gallate, flavonoids, Dehydrotrametenolic acid, 6-Shogaol and combinations thereof.

According to an embodiment, said at least one TRP channel modulator is selected from the group consisting of Capsaicin, capsaicinoid, capsinoid, Capsiate, Dihydrocapsiate, Nordihydrocapsiate, Piperine, Eugenol, Resiniferatoxin, Gingerol, Shogaol, Zingerone, Paradol, Grifolin, Neogrifolin, Albaconol, Scutigeral, Cochinchinenin, Loureirin, Allyl isothiocyanate, Cinnamaldehyde, Allicin, Diallyl disulphide, Ligustilide, Dehydroligustilide, Alpha-spinasterol, Guaiacol, Methyl salicylate, Vanillin, Methyl syringate, Resveratrol, Pinosylvin methyl ether, Salidroside, Bisandrographolide, Ginsenoside, Triptolide, Evodiamine, Nicotine, Yohimbine, Hydroxy-alphasanshool, Artepillin, Thapsigargin, Ricinoleic acid, Incensole acetate, Hyperforin, Vanillotoxins, Quinine, Waixenicin, Citral, Camphor, Carvacrol, Thymol, Menthol, cineole, Perillaldehyde, Perillaketone, Polygodial, Drimanial, Isovelleral, Cinnamodial, Cinnamosmolide, Cinnamolide, Warburganal, Scalaradial, Aframodial, Ancistrodial, Merulidial, Drimenol, Umbellulone, Cineole, Carveol, 6-tert-butyl-m-cresol, Dihydrocarveol, Borneol, Perillaldehyde, Perillaketone, oleoylethanolamide, N-oleoyldopamine, 3-methyl-N-oleoyldopamine, oleamide, 1-monoacylglycerol, 2-monoacylglycerol, 1-Arachidonoylglycerol, 2-arachidonoylglycerol, miogadial, miogatrial, polygodial, terpenes with an alpha,beta-unsaturated 1,4-dialdehyde moiety, sanshool, evodiamine, acesulfame-K, cyclamate, CuSO₄, ZnSO₄, FeSO₄, arvanil, N-arachidonoyl-dopamine, flufenamic acid dopamide, dopamine amide of fenamic acid, 4-hydroxynonenal, 1-[2-(1-adamantyl)ethyl]-1-pentyl-3-[3-(4-pyridyl)propyl]urea, N-palmitoylethanolamide (PEA) and N-oleoylethanolamide (OEA) and combinations thereof.

According to an embodiment, said at least one TRP channel modulator is a CB1 receptor agonist.

According to an embodiment, said pharmaceutical composition comprises at least one terpene selected from the group consisting of pinene, limonene, linalool, caryophyllene, caryophyllene oxide, myrcene, humulene, borneol, eucalyptol, terpineol, nerolidol, phytol, geraniol, bisabolol, camphene, beta-amyrin, thuj one, citronellol, pulegone, cycloartenol, cymene, sabinene, carene, terpinene, fenchol, isopulegol, guaiol, phellandrene, eudesmol, ocimene, cardinene, elemene, gurjunene, farnesene, friedelin, carvacrol, eugenol, camphor, menthol, iso-menthone, neral, gerial, viridiflorol, germacrene, thymol, Menth-2-en-1-ol, farensol, carotol, myrtenol and combinations thereof. According to some such embodiments, the pharmaceutical composition comprises at least one, at least two, at least three, at least four or at least five such terpenes. In some such embodiments, the terpene does not function as a modulator.

According to an embodiment, the composition further comprises an additive selected from the group consisting of antioxidants, emulsifiers and texturizers vegetable oils, plant extracts, honey, pharmaceutical excipients, sucrose, glucose and fructose, pharmaceutical excipients and combinations thereof. According to an embodiment, the composition comprises a surfactant selected from the group consisting of phospholipids, glycerides, glycolipids and combinations thereof. According to an embodiment, the composition further comprises a food-approved texturizer. According to an embodiment, the composition further comprises at least loppm ethanol. According to an embodiment, the composition further comprises at least one of vitamin C, vitamin E, polyunsaturated fatty acids, beeswax and coconut oil. According to an embodiment, the composition further comprises a sweetener.

According to an aspect of some embodiments of the present invention, further provided is a pharmaceutical preparation comprising the pharmaceutical composition as disclosed herein.

According to an embodiment, said preparation comprises cannabidiol and said preparation comprises between 0.1 and 100 milligram TRP channel modulator. According to an embodiment, said preparation comprises cannabidiol and said preparation comprises between 0.1 and 100 milligram TRP channel modulator. In some such embodiments, said preparation comprises between 0.1 and 100 milligram TRP channel modulator, such as at least 0.1, about 0.2, about 0.3, about 0.4, about 0.5, about 0.6, about 0.7, about 0.8, about 0.9, about 1, about 2, about 3, about 4, about 5, about 6, about 7, about 8, about 9, about 10, about 20, about 30, about 40, about 50, about 60, about 70, about 80, about 90 and up to 100 miligram TRP channel modulator.

According to an embodiment, said preparation comprises cannabidiol and said preparation comprises between 0.1 and 100 milligram PPAR modulator. According to an embodiment, said preparation comprises cannabidiol and said preparation comprises between 0.1 and 100 milligram PPAR channel modulator. In some such embodiments, said preparation comprises between 0.1 and 100 milligram PPAR channel modulator, such as at least 0.1, about 0.2, about 0.3, about 0.4, about 0.5, about 0.6, about 0.7, about 0.8, about 0.9, about 1, about 2, about 3, about 4, about 5, about 6, about 7, about 8, about 9, about 10, about 20, about 30, about 40, about 50, about 60, about 70, about 80, about 90 and up to 100 miligram TRP channel modulator.

According to an embodiment, the preparation comprises the composition in a dosage form suitable for oral administration, such as, for example, as a pill, tablet, capsule, syrup, solution, suspension, powder or the like, or a combination thereof.

According to an embodiment, the preparation comprises the composition in a form suitable for administration by inhalation, such as, for example, in an aerosol, inhaler, nebulizer, vaporizer or the like, or combinations thereof.

According to an embodiment, the preparation comprises the composition in a form suitable for parenteral administration, such as for example, for administration by intradermal, subcutaneous, intramuscular, intraosseous, intraperitioneal or intravenous injection, or any combination thereof.

According to an embodiment, the preparation comprises a suppository, such as a vaginal suppository (including a douche, pessary or the like), a rectal suppository, urethral suppository or nasal suppository, or any combination thereof.

According to an embodiment, the preparation comprises a cigarette.

According to an embodiment, the pharmaceutical preparation comprises cannabidiol. In some such embodiments, said preparation comprises between 0.1 and 100 milligram cannabidiol, such as at least 0.1, about 0.2, about 0.3, about 0.4, about 0.5, about 0.6, about 0.7, about 0.8, about 0.9, about 1, about 2, about 3, about 4, about 5, about 6, about 7, about 8, about 9, about 10, about 20, about 30, about 40, about 50, about 60, about 70, about 80, about 90 and up to 100 miligram cannabidiol.

According to an aspect of some embodiments of the present invention, there is provided the pharmaceutical composition or the pharmaceutical preparation as disclosed herein for use in treating a condition or a symptom thereof, wherein said condition is selected from the group consisting of inflammation, an immune disease, an autoimmune disease or a combination thereof.

According to an embodiment, the condition is characterized by anandamide deficiency.

According to an embodiment, the condition is selected from the group consisting of autoimmune diseases, autoimmune hepatitis, liver inflammation, Cirrhuosis and combinations thereof.

According to an embodiment, further provided is a method for treating a condition or a symptom thereof in a subject in need thereof, wherein said condition is selected from the group consisting of inflammation, an immune disease, an autoimmune disease or a combination thereof, the method comprising administering to said subject a pharmaceutically effective amount of the pharmaceutical composition or the pharmaceutical preparation as disclosed herein.

According to an embodiment, further provided is a method for treating a condition or a symptom thereof in a subject in need thereof, wherein said condition involves inflammation, an immune disease, an autoimmune disease or a combination thereof, the method comprising administering to said subject a modulator composition comprising a pharmaceutically effective amount of at least one modulator selected from the group consisting of a TRP channel modulator and a PPAR modulator, wherein CBD is not present as a sole modulator; and administering a pharmaceutically effective amount of a steroid.

According to an embodiment, said method comprises chronically administering to said subject a pharmaceutically effective amount of said steroid and a pharmaceutically effective amount of a PPAR modulator. According to an embodiment, said pharmaceutically effective amount of said steroid is smaller than the amount of steroid required to achieve the same result without administering said PPAR modulator.

According to an embodiment, said method comprises chronically administering to said subject said modulator composition and a pharmaceutically effective amount of said steroid and a pharmaceutically effective amount of a TRP channel modulator. According to an embodiment, said pharmaceutically effective amount of said steroid is smaller than the amount of steroid required to ahieve the same result without administering said TRP channel modulator.

According to an embodiment, said subject is treated with steroid and suffers from a side-effect of the steroid.According to an embodiment, said condition is selected from the group consisting of autoimmune diseases, autoimmune hepatitis, liver inflammation, Cirrhuosis and combinations thereof.

According to an embodiment, the method comprises administering to said patient cannabidiol and at least one additional modulator selected from the group consisting of a TRP channel modulator and a PPAR modulator.

According to an embodiment, said modulator comprises cannabidiol. According to an embodiment, said at least one modulator and said cannabidiol are administered in separate compositions. According to an embodiment, said at least one modulator and said cannabidiol are administered together in a single composition.

According to an embodiment, the at least one modulator and the cannabidiol are administered independently, sequentially, simultaneously or concomitantly in separate compositions. In some embodiments, wherein the at least one modulator and the cannabidiol are administered sequentially, the at least one modulator is administered first, followed by the cannabidiol. In some embodiments, wherein the at least one modulator and the cannabidiol are administered sequentially, the cannabidiol is administered first, followed by the at least one modulator.

According to an embodiment, said modulator comprises at least one TRP channel modulator.

According to an embodiment, said modulator comprises at least two TRP channel modulators.

According to an embodiment, said modulator comprises modulators of at least two different TRP channels.

According to an embodiment, said modulator comprises at least two different modulators of a same TRP channel.

According to an embodiment, said modulator composition comprises a TRV channel modulator, such as a TRPV1 channel modulator.

According to an embodiment, said at least one TRP channel modulator is selected from the group consisting of a TRPA1 modulator and a TRPM8 modulator, or combinations thereof. According to an embodiment, said modulator comprises at least one PPAR modulator.

According to an embodiment, said at least one PPAR modulator comprises at least two PPAR modulators.

According to an embodiment, said at least one PPAR modulator modulators of at least two different PPARs.

According to an embodiment, said at least one PPAR modulator comprises two different modulators of a same PPAR.

According to an embodiment, said at least one PPAR modulator comprises at least one PPAR-γ modulator modulator.

According to an embodiment, said modulator comprises at least one TPR channel modulator and at least one PPAR modulator. According to an embodiment, said at least one PPAR modulator and said at least one TRP channel modulator are administered in separate compositions.

According to an embodiment, said at least one PPAR modulator and said at least one TRP channel modulator are administered in a single composition.

According to an embodiment, the at least one PPAR modulator and the TRP channel modulator are administered independently, sequentially, simultaneously or concomitantly in separate compositions. In some embodiments, wherein the at least one PPAR modulator and the TRP channel modulator are administered sequentially, the at least one PPAR modulator is administered first, followed by the TRP channel modulator. In some embodiments, wherein the at least one PPAR modulator and the TRP channel modulator are administered sequentially, the TRP channel modulator is administered first, followed by the at least one PPAR modulator.

According to an embodiment, said modulator composition comprises at least one TRP channel modulator and at least two PPAR modulators.

According to an embodiment, said modulator composition comprises at least one TRP channel modulator and modulators of at least two different PPARs.

According to an embodiment, said modulator composition comprises at least one TRP channel modulator and at least two different modulators of a same PPAR.

According to an embodiment, said modulator composition comprises at least one TRP channel modulator and at least one PPAR-γ modulator.

According to an embodiment, said modulator comprises at least one PPAR modulator and at least two TRP channel modulators.

According to an embodiment, said modulator comprises at least one PPAR modulator and at least two TRP channel modulators.

According to an embodiment, said modulator comprises at least two TPR channel modulator and at least two PPAR modulators.

According to an embodiment, said modulator comprises tetrahydrocannabinol. According to an embodiment, said modulator comprises cannabidiol and at least one additional modulator selected from the group consisting of a TRP channel modulator and a PPAR modulator. According to an embodiment, said modulator comprises cannabidiol and tetrahydrocannabinol, wherein a cannabidiol to tetrahydrocannabinol weight per weight ratio is greater than 3:1 such as at least 3:1, at least 4:1, at least 5:1, at least 6:1, at least 7:1, at least 8:1, at least 9:1 or at least 10:1.

According to an embodiment, said at least one modulator is a cannabinoid.

According to an embodiment, said at least one TRP channel modulator is a terpene.

According to an embodiment, said at least one PPAR modulator is selected from the group consisting of Beta Caryophyllene, Pinene, Carvacrol, Geraniol, Farnesol, geranylgeraniol, Phytol, Phytanic acid, Abietic acid, Auraptene, bixin, norbixin, Amorfrutins, Formononetin, (−)-Catechin, THC, (9S,13R)-12-Oxo-phytodienoic acid, Hydroxy unsaturated fatty acids, Commipheric acid, Kaempferol-3-O-β-glucopyranoside, Citral, Alkamides, Tocotrienols, Deoxyelephantopin, Acylated flavonol glycosides, Kaempferol, quercetin, Genistein, 5′-Formylglabridin, (2R,3R)-3,4′,7-trihydroxy-3′-prenylflavane, echinatin, (3R)-2′,3′,7-trihydroxy-4′-methoxyisoflavan, kanzonol X, kanzonol W shinpterocarpin, licoflavanone A, glabrol, shinflavanone, gancaonin L, glabrone, Licochalcone E, Flavonoids and 3-arylcoumarins, Meranzin, Magnolol, honokiol, Lunularin, Cucurbitane-type triterpene glycosides, Polyacetylenes, Biochanin A, Ginsenoside 20(S)-protopanaxatriol, ginsenoside Rb₁, Oleanonic acid, Pseudolaric acid B, Daidzein, Amorphastilbol, Carnosic acid, carnosol, 12-O-methyl carnosic acid, α-linolenic acid, α-Linolenic acid, linoleic acid, naringenin, Saurufuran A, Isosilybin A, Gallotannins, Isoflavones, Ellagic acid, epicatechin gallate, flavonoids, Dehydrotrametenolic acid, 6-Shogaol and combinations thereof.

According to an embodiment, said at least one TRP channel modulator is selected from the group consisting of Capsaicin, capsaicinoid, capsinoid, Capsiate, Dihydrocapsiate, Nordihydrocapsiate, Piperine, Eugenol, Resiniferatoxin, Gingerol, Shogaol, Zingerone, Paradol, Grifolin, Neogrifolin, Albaconol, Scutigeral, Cochinchinenin, Loureirin, Allyl isothiocyanate, Cinnamaldehyde, Allicin, Diallyl disulphide, Ligustilide, Dehydroligustilide, Alpha-spinasterol, Guaiacol, Methyl salicylate, Vanillin, Methyl syringate, Resveratrol, Pinosylvin methyl ether, Salidroside, Bisandrographolide, Ginsenoside, Triptolide, Evodiamine, Nicotine, Yohimbine, Hydroxy-alphasanshool, Artepillin, Thapsigargin, Ricinoleic acid, Incensole acetate, Hyperforin, Vanillotoxins, Quinine, Waixenicin, Citral, Camphor, Carvacrol, Thymol, Menthol, cineole, Perillaldehyde, Perillaketone, Polygodial, Drimanial, Isovelleral, Cinnamodial, Cinnamosmolide, Cinnamolide, Warburganal, Scalaradial, Aframodial, Ancistrodial, Merulidial, Drimenol, Umbellulone, Cineole, Carveol, 6-tert-butyl-m-cresol, Dihydrocarveol, Borneol, Perillaldehyde, Perillaketone, oleoylethanolamide, N-oleoyldopamine, 3-methyl-N-oleoyldopamine, oleamide, 1-monoacylglycerol, 2-monoacylglycerol, 1-Arachidonoylglycerol, 2-arachidonoylglycerol, miogadial, miogatrial, polygodial, terpenes with an alpha,beta-unsaturated 1,4-dialdehyde moiety, sanshool, evodiamine, acesulfame-K, cyclamate, CuSO₄, ZnSO₄, FeSO₄, arvanil, N-arachidonoyl-dopamine, flufenamic acid dopamide, dopamine amide of fenamic acid, 4-hydroxynonenal, 1-[2-(1-adamantyl)ethyl]-1-pentyl-3-[3-(4-pyridyl)propyl]urea, N-palmitoylethanolamide (PEA) and N-oleoylethanolamide (OEA) and combinations thereof.

According to an embodiment, said at least one TRP channeel modulator is a CB1 receptor agonist.

According to an embodiment, said modulator composition further comprises at least one terpene selected from the group consisting of pinene, limonene, linalool, caryophyllene, caryophyllene oxide, myrcene, humulene, borneol, eucalyptol, terpineol, nerolidol, phytol, geraniol, bisabolol, camphene, beta-amyrin, thujone, citronellol, pulegone, cycloartenol, cymene, sabinene, carene, terpinene, fenchol, isopulegol, guaiol, phellandrene, eudesmol, ocimene, cardinene, elemene, gurjunene, farnesene, friedelin, carvacrol, eugenol, camphor, menthol, iso-menthone, neral, gerial, viridiflorol, germacrene, thymol, Menth-2-en-1-ol, farensol, carotol, myrtenol and combinations thereof. According to some such embodiments, the pharmaceutical composition comprises at least one, at least two, at least three, at least four or at least five such terpenes. In some such embodiments, the terpene does not function as a modulator.

The scope of the invention shall include all modifications and variations that may fall within the scope of the attached claims. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A pharmaceutical composition comprising a carrier and a pharmaceutically effective amount of a steroid and a pharmaceutically effective amount of at least one modulator selected from the group consisting of at least one transient receptor potential (TRP) channel modulator, at least one peroxisome proliferator-activated receptor (PPAR) modulator and a combination thereof, wherein cannabidiol (CBD) is not present as a sole modulator.

2. (canceled)

3. The pharmaceutical composition of claim 1, wherein said at least one TRP channel modulator comprises at least two different TRP channel modulators.

4. (canceled)

5. The pharmaceutical composition of claim 1, wherein said at least one TRP channel modulator comprises a transient receptor potential vanilloid (TRPV) channel modulator.

6-7. (canceled)

8. The pharmaceutical composition claim 1, wherein said at least one PPAR modulator comprises a peroxisome proliferator-activated receptor-γ (PPAR-γ) modulator.

9. The pharmaceutical composition of claim 1, wherein said at least one PPAR modulator comprises at least two different PPAR modulators.

10. (canceled)

11. The pharmaceutical composition of claim 1, further comprising at least one selected from the group consisting of cannabidiol, tetrahydrocannabinol and a combination thereof.

12. (canceled)

13. The pharmaceutical composition of claim 1, comprising cannabidiol and tetrahydrocannabinol, wherein a cannabidiol to tetrahydrocannabinol weight per weight ratio is greater than 3:1.

14. The pharmaceutical composition of claim 1, wherein said at least one modulator is selected from the group consisting of a cannabinoid, a terpene and a combination thereof.

15. (canceled)

16. The pharmaceutical composition of claim 1, wherein said at least one PPAR modulator is selected from the group consisting of Beta Caryophyllene, Pinene, Carvacrol, Geraniol, Farnesol, geranylgeraniol, Phytol, Phytanic acid, Abietic acid, Auraptene, bixin, norbixin, Amorfrutins, Formononetin, (−)-Catechin, THC, (9S,13R)-12-Oxo-phytodienoic acid, Hydroxy unsaturated fatty acids, Commipheric acid, Kaempferol-3-O-β-glucopyranoside, Citral, Alkamides, Tocotrienols, Deoxyelephantopin, Acylated flavonol glycosides, Kaempferol, quercetin, Genistein, 5′-Formylglabridin, (2R,3R)-3,4′,7-trihydroxy-3′-prenylflavane, echinatin, (3R)-2′,3′,7-trihydroxy-4′-methoxyisoflavan, kanzonol X, kanzonol W shinpterocarpin, licoflavanone A, glabrol, shinflavanone, gancaonin L, glabrone, Licochalcone E, Flavonoids and 3-arylcoumarins, Meranzin, Magnolol, honokiol, Lunularin, Cucurbitane-type triterpene glycosides, Polyacetylenes, Biochanin A, Ginsenoside 20(S)-protopanaxatriol, ginsenoside Oleanonic acid, Pseudolaric acid B, Daidzein, Amorphastilbol, Carnosic acid, carnosol, 12-O-methyl carnosic acid, α-linolenic acid, α-Linolenic acid, linoleic acid, naringenin, Saurufuran A, Isosilybin A, Gallotannins, Isoflavones, Ellagic acid, epicatechin gallate, flavonoids, Dehydrotrametenolic acid, 6-Shogaol and combinations thereof.

17. The pharmaceutical composition of claim 1, wherein said at least one TRP channel modulator is selected from the group consisting of Capsaicin, capsaicinoid, capsinoid, Capsiate, Dihydrocapsiate, Nordihydrocapsiate, Piperine, Eugenol, Resiniferatoxin, Gingerol, Shogaol, Zingerone, Paradol, Grifolin, Neogrifolin, Albaconol, Scutigeral, Cochinchinenin, Loureirin, Allyl isothiocyanate, Cinnamaldehyde, Allicin, Diallyl disulphide, Ligustilide, Dehydroligustilide, Alpha-spinasterol, Guaiacol, Methyl salicylate, Vanillin, Methyl syringate, Resveratrol, Pinosylvin methyl ether, Salidroside, Bisandrographolide, Ginsenoside, Triptolide, Evodiamine, Nicotine, Yohimbine, Hydroxy-alphasanshool, Artepillin, Thapsigargin, Ricinoleic acid, Incensole acetate, Hyperforin, Vanillotoxins, Quinine, Waixenicin, Citral, Camphor, Carvacrol, Thymol, Menthol, cineole, Perillaldehyde, Perillaketone, Polygodial, Drimanial, Isovelleral, Cinnamodial, Cinnamosmolide, Cinnamolide, Warburganal, Scalaradial, Aframodial, Ancistrodial, Merulidial, Drimenol, Umbellulone, Cineole, Carveol, 6-tert-butyl-m-cresol, Dihydrocarveol, Borneol, Perillaldehyde, Perillaketone, oleoylethanolamide, N-oleoyldopamine, 3-methyl-N-oleoyldopamine, oleamide, 1-monoacylglycerol, 2-monoacylglycerol, 1-Arachidonoylglycerol, 2-arachidonoylglycerol, miogadial, miogatrial, polygodial, terpenes with an alpha,beta-unsaturated 1,4-dialdehyde moiety, sanshool, evodiamine, acesulfame-K, cyclamate, CuSO4, ZnSO4, FeSO4, arvanil, N-arachidonoyl-dopamine, flufenamic acid dopamide, dopamine amide of fenamic acid, 4-hydroxynonenal, 1-[2-(1-adamantyl)ethyl]-1-pentyl-3-[3-(4-pyridyl)propyl]urea, N-palmitoylethanolamide (PEA) and N-oleoylethanolamide (OEA) and combinations thereof.

18. The pharmaceutical composition of claim 1, wherein said at least one TRP channel modulator is a cannabinoid receptor type 1 (CB1) receptor agonist.

19. The pharmaceutical composition of claim 1, further comprising at least one terpene selected from the group consisting of pinene, limonene, linalool, caryophyllene, caryophyllene oxide, myrcene, humulene, borneol, eucalyptol, terpineol, nerolidol, phytol, geraniol, bisabolol, camphene, beta-amyrin, thuj one, citronellol, pulegone, cycloartenol, cymene, sabinene, carene, terpinene, fenchol, isopulegol, guaiol, phellandrene, eudesmol, ocimene, cardinene, elemene, gurjunene, farnesene, friedelin, carvacrol, eugenol, camphor, menthol, iso-menthone, neral, gerial, viridiflorol, germacrene, thymol, Menth-2-en-1-ol, farensol, carotol, myrtenol and combinations thereof.

20. A pharmaceutical preparation comprising the pharmaceutical composition of claim 1.

21. The pharmaceutical preparation of claim 20, wherein said preparation comprises cannabidiol and wherein said preparation comprises between 0.1 and 100 milligram TRP channel modulator and/or between 0.1 and 100 milligram PPAR channel modulator.

22-25. (canceled)

26. A method for treating a condition or a symptom thereof in a subject in need thereof, wherein said condition is selected from the group consisting of inflammation, an immune disease, an autoimmune disease or a combination thereof, the method comprising administering to said subject a pharmaceutically effective amount of the composition of claim 1.

27. A method for treating a condition or a symptom thereof in a subj ect in need thereof wherein said condition involves inflammation, an immune disease, an autoimmune disease or a combination thereof, the method comprising administering to said subject a pharmaceutically effective amount of at least one modulator selected from the group consisting of a TRP channel modulator and a PPAR modulator, wherein CBD is not present as a sole modulator; and administering a pharmaceutically effective amount of a steroid.

28-30. (canceled)

31. The method of claim 27, wherein said condition is selected from the group consisting of autoimmune diseases, autoimmune hepatitis, liver inflammation, cirrhosis and combinations thereof.

32. The method of claim 27, comprising administering to said patient cannabidiol and at least one additional modulator selected from the group consisting of a TRP channel modulator, a PPAR modulator and a combination thereof.

33-46. (canceled)

47. The method of claim 27, wherein said at least one PPAR modulator is selected from the group consisting of Beta Caryophyllene, Pinene, Carvacrol, Geraniol, Farnesol, geranylgeraniol, Phytol, Phytanic acid, Abietic acid, Auraptene, bixin, norbixin, Amorfrutins, Formononetin, (−)-Catechin, THC, (9S,13R)-12-Oxo-phytodienoic acid, Hydroxy unsaturated fatty acids, Commipheric acid, Kaempferol-3-O-β-glucopyranoside, Citral, Alkamides, Tocotrienols, Deoxyelephantopin, Acylated flavonol glycosides, Kaempferol, quercetin, Genistein, 5′-Formylglabridin, (2R,3R)-3,4′,7-trihydroxy-3′-prenylflavane, echinatin, (3R)-2′,3′,7-trihydroxy-4′-methoxyisoflavan, kanzonol X, kanzonol W shinpterocarpin, licoflavanone A, glabrol, shinflavanone, gancaonin L, glabrone, Licochalcone E, Flavonoids and 3-arylcoumarins, Meranzin, Magnolol, honokiol, Lunularin, Cucurbitane-type triterpene glycosides, Polyacetylenes, Biochanin A, Ginsenoside 20(S)-protopanaxatriol, ginsenoside Rb₁, Oleanonic acid, Pseudolaric acid B, Daidzein, Amorphastilbol, Carnosic acid, carnosol, 12-O-methyl carnosic acid, α-linolenic acid, α-Linolenic acid, linoleic acid, naringenin, Saurufuran A, Isosilybin A, Gallotannins, Isoflavones, Ellagic acid, epicatechin gallate, flavonoids, Dehydrotrametenolic acid, 6-Shogaol and combinations thereof.

48. The method of claim 27, wherein said at least one TRP channel modulator is selected from the group consisting of Capsaicin, capsaicinoid, capsinoid, Capsiate, Dihydrocapsiate, Nordihydrocapsiate, Piperine, Eugenol, Resiniferatoxin, Gingerol, Shogaol, Zingerone, Paradol, Grifolin, Neogrifolin, Albaconol, Scutigeral, Cochinchinenin, Loureirin, Allyl isothiocyanate, Cinnamaldehyde, Allicin, Diallyl disulphide, Ligustilide, Dehydroligustilide, Alpha-spinasterol, Guaiacol, Methyl salicylate, Vanillin, Methyl syringate, Resveratrol, Pinosylvin methyl ether, Salidroside, Bi sandrographolide, Ginsenoside, Triptolide, Evodiamine, Nicotine, Yohimbine, Hydroxy-alphasanshool, Artepillin, Thapsigargin, Ricinoleic acid, Incensole acetate, Hyperforin, Vanillotoxins, Quinine, Waixenicin, Citral, Camphor, Carvacrol, Thymol, Menthol, cineole, Perillaldehyde, Perillaketone, Polygodial, Drimanial, Isovelleral, Cinnamodial, Cinnamosmolide, Cinnamolide, Warburganal, Scalaradial, Aframodial, Ancistrodial, Merulidial, Drimenol, Umbellulone, Cineole, Carveol, 6-tert-butyl-m-cresol, Dihydrocarveol, Borneol, Perillaldehyde, Perillaketone, oleoylethanolamide, N-oleoyldopamine, 3-methyl-N-oleoyldopamine, oleamide, 1-monoacylglycerol, 2-monoacylglycerol, 1-Arachidonoylglycerol, 2-arachidonoylglycerol, miogadial, miogatrial, polygodial, terpenes with an alpha,beta-unsaturated 1,4-dialdehyde moiety, sanshool, evodiamine, acesulfame-K, cyclamate, CuSO4, ZnSO4, FeSO4, arvanil, N-arachidonoyl-dopamine, flufenamic acid dopamide, dopamine amide of fenamic acid, 4-hydroxynonenal, 1-[2-(1-adamantyl)ethyl]-1-pentyl-3-[3-(4-pyridyl)propyl]urea, N-palmitoylethanolamide (PEA) and N-oleoylethanolamide (OEA) and combinations thereof.

49-50. (canceled)
